(19) United States
(12) Patent Application Publication
(10) Pub. No.: US 2009/0203773 A1
(43) Pub. Date: Aug. 13, 2009

(54) Compounds for the Treatment of Non-Autoimmune Type 2 Diabetes Mellitus and/or Syndrome X

(76) Inventors: Daniel D'Orazio, Halten (CH); Antoine De Saizieu, Brunstatt (FR); Goede Schuler, Eimeldingen (DE); Ying Wang-Schmidt, Stallikon (CH); Christof Wehrli, Witterswil (CH); Swen Wolfram, Waldshut-Tiengen (DE); Karin Wertz, Rheinfelden (DE); Daniel Raederstorff, Flaxlanden (DE)

Correspondence Address: NIXON & VANDERHYE, PC, 901 NORTH GLEBE ROAD, 11TH FLOOR, ARLINGTON, VA, 22203, US

(21) Appl. No.: 11/922,604

(22) PCT Filed: Jun. 23, 2006
(86) PCT No.: PCT/EP2006/006049

§ 371 Date: Apr. 14, 2008

(30) Foreign Application Priority Data
Jun. 24, 2005 (EP) 05 013 661.3

(51) Int. Cl. A61K 31/357; C07D 317/44; C07C 69/76; A61K 31/235; A61P 3/10

(52) U.S. Cl. 514/464; 549/437; 560/8; 514/532

(57) ABSTRACT

The present invention relates to compounds of the formula (I); wherein R¹ is H, CH₃ or OCH₃; R³=H, OH, CH₃, OCH₃, O-glucose or benzoyloxy; R⁴=H; R⁵=H or OH; R⁶=H or OCH₃; R⁷=H, CH₃, OCH₃, cinnamoyloxy or (3,4,5-trimethoxy)-benzoyloxy; R⁸=H, OH, CH₃ or OCH₃; or R⁷ and R⁸ form together a group 0-CH₂—O; R⁹=H or OCH₃; R¹⁰=H or N-acetyl, N-methyl-2-aminoethyl; to their use as medicament for the treatment of non-autoimmune type 2 diabetes mellitus and/or syndrome X, to dietary and pharmaceutical compositions containing them and to a method for the treatment of non-autoimmune type 2 diabetes mellitus and/or syndrome X in animals including humans, said method comprising the step of administering an effective dose of a compound of the formula (I) to animals including humans which are in need thereof.

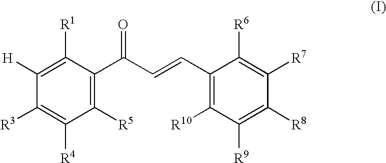

The present invention relates to compounds of the formula I as defined below

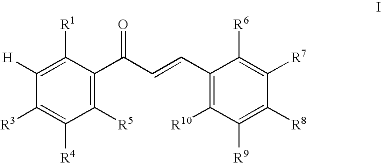

for use as medicament for the treatment of non-autoimmune type 2 diabetes mellitus and/or syndrome X, to dietary and pharmaceutical compositions containing them and to a method for the treatment of non-autoimmune type 2 diabetes mellitus and/or syndrome X in animals including humans, said method comprising the step of administering an effective dose of a compound of the formula I to animals including humans which are in need thereof. The present invention further relates to compounds of the formulae I, especially to compounds I-2, I-7, I-8, I-9, I-13, I-14, I-15, I-17 and I-19, as defined below and their use as medicament.

In the context of this invention “treatment” also encompasses co-treatment as well as prevention and control.

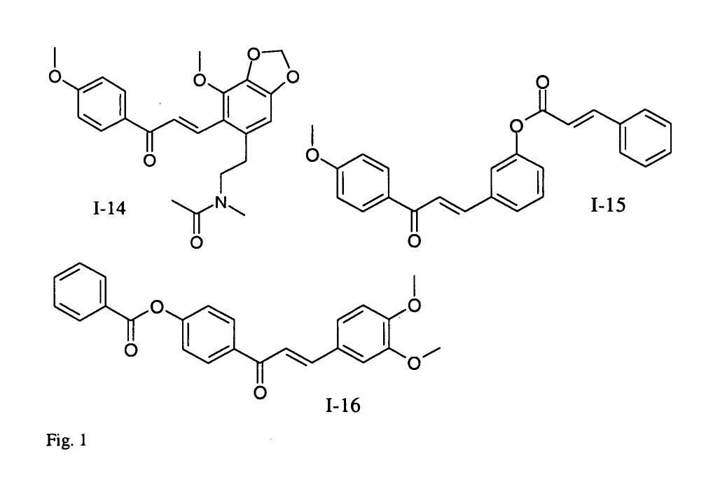

The most preferred embodiments of the invention are shown in FIG. 1.

Animals in the context of the present invention may be mammals including humans.

Preferred examples of mammals beside humans are dogs, cats, guinea pigs, (jack) rabbits, hares, ferrets, horses, and ruminants (cattle, sheep and goat).

Diabetes mellitus defines a complex of metabolic diseases derived from multiple causative factors and is characterized by impaired glucose metabolism, usually associated with impaired protein and fat metabolism. This results in elevated fasting and postprandial serum glucose level that leads to complications if left untreated.

Four different forms of diabetes mellitus are known, (1) type 1 diabetes mellitus, (2) type 2 diabetes mellitus, (3) the so-called gestational diabetes mellitus, which begins or is recognized for the first time during pregnancy, and (4) some other forms which are mainly based on genetic defects. The two major forms of diabetes mellitus are the type 1 and type 2 diabetes mellitus, of which type 2 diabetes mellitus is the most prevailing form.

Type 2 diabetes mellitus is associated with hyperglycemia, hypercholesterolemia and hyperlipidemia. The insensitivity to insulin in type 2 diabetes mellitus leads to a decrease in glucose utilization by the liver, muscle and the adipose tissue and to an increased blood glucose level. Uncontrolled hyperglycemia is associated with the dysfunction and failure of various organs such as the eyes, heart, blood vessels, kidney and nerves thus leading to increased and premature mortality due to an increased risk for microvascular and macrovascular diseases, including nephropathy, neuropathy, retinopathy, ulceration of the legs and feet, fatty liver disease, hypertension, cardiovascular diseases, and cerebrovascular diseases (stroke), the so-called diabetic complications. Recent evidence showed that tight glycemic control is a major factor in the prevention of these complications in type 2 diabetes mellitus. Therefore, optimal glycemic control by drugs or therapeutic regimens is an important approach for the treatment of type 2 diabetes mellitus.

Type 2 diabetes mellitus is the form of diabetes mellitus which occurs predominantly in adults, in whom adequate production of insulin is available for use in the early stage of the diseases, yet a defect exists in insulin action especially insulin-mediated utilization and metabolism of glucose in peripheral tissues. The changes in various tissues associated with type 2 diabetes mellitus exist even before clinical symptoms are detected.

Therapy of type 2 diabetes mellitus initially involves dietary and lifestyle changes. When these measures fail to maintain adequate glycemic control, the patients are treated with oral hypoglycemic agents and/or exogenous insulin. The current oral pharmacological agents for the treatment of type 2 diabetes mellitus include those that potentiate insulin secretion (sulphonylurea agents), those that improve the action of insulin in the liver (biguanide agents), insulin sensitizing agents (thiazolidinediones) and agents which act to inhibit the uptake of glucose in the gastrointestinal tract (α-glucosidase inhibitors). However, currently available agents generally fail to maintain adequate glycemic control in the long term due to progressive deterioration in hyperglycemia, resulting from progressive loss of pancreatic cell function. The proportion of patients able to maintain target glycemic levels decreases markedly overtime necessitating the administration of additional/alternative pharmacological agents. Furthermore, the drugs may have unwanted side effects and are associated with high primary and secondary failure rates.

Therefore, there is a need for compounds with minimal side effects for the prevention, control and/or treatment of type 2 diabetes mellitus and for the prevention of the physical complications associated with it as mentioned above. Many patients are interested in alternative therapies which could minimize the side effects associated with high-dose of drugs and yield additive clinical benefits. Type 2 diabetes mellitus is a progressive and chronic disease, which usually is not recognized until significant damage has occurred to the pancreatic cells responsible for producing insulin and to the cardiovascular system. Therefore, there is also an increasing interest in the development of a dietary supplement that may be used to prevent the development of type 2 diabetes mellitus in people at risk especially in elderly persons, but also in obese children, who are at high risk for developing type 2 diabetes mellitus. Since type 2 diabetes mellitus is often associated with symptoms from syndrome X (“metabolic syndrome”), such as hypertriglyceridemia or dyslipidemia, the compounds according to the present invention are also useful for the treatment or prevention of syndrome X.

We now found that compounds of the formula I

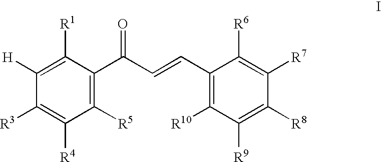

wherein R¹ is H, CH₃ or OCH₃;
R³=H, OH, CH₃, OCH₃, O-glucose or benzoyloxy;

### R⁴=H;

### R⁵=H or OH;

### R⁶=H or OCH₃;

R⁷=H, CH₃, OCH₃, cinnamoyloxy or (3,4,5-trimethoxy)-benzoyloxy;

### R⁸=H, OH, CH₃ or OCH₃;

or R⁷ and R⁸ form together a group O—CH₂—O;

### R⁹=H or OCH₃;

R¹⁰=H or N-acetyl, N-methyl-2-aminoethyl;
may be effective agents in the prevention, control and/or treatment of non-autoimmune type 2 diabetes mellitus and/or syndrome X, in animals including humans, especially in mammals including humans, so that they can be used therefor.

Therapeutic effects of these compounds may include, but are not limited to, the following ones. Therefore, the present invention is directed to the use of the compounds of the formula I as defined above for
helping to manage blood sugar levels, i.e. helping the body by balancing the blood sugar levels; helping to keep balanced blood glucose levels, particularly in humans with diabetes; aiding by enhancing the glucose uptake by the cells and by reducing sugar levels, thus improving or restoring the glucose tolerance; lowering the blood glucose level; optimizing the glycemic response; normalizing the glucose tolerance; i.e. the compounds of the formula I may be α-glucosidase inhibitors, hyperglycemia treating and/or controlling agents and blood glucose lowering agents;
reducing sweetness cravings;
preserving or improving the pancreatic β-cell function, thus promoting a healthy pancreatic function; i.e. the compounds of the formula I are pancreatic β-cell function improvers;
treating or controlling the insulin sensitivity by e.g. helping to restore/enhance the insulin sensitivity; i.e. the compounds of the formula I may be insulin sensitizing agents;
delaying, preventing or controlling non-autoimmune type 2 diabetes mellitus and thus preventing also the diabetes accompanying disorders/complications such as the ones mentioned above, i.e. the compounds of the formula I may be diabetes type 2 preventing agents;
activating adipocytes thus increasing insulin sensitivity;
repartioning of fat from lipolytic visceral fat depots into subcutaneous fat thus decreasing the risk of obesity associated pathologies such as cardiovascular diseases;
reducing the circulation of free fatty acids (FFA), thus improving insulin sensitivity in obese persons;
maintaining endothelial function;
lowering triglyceride levels in the blood; maintaining a healthy/normal blood lipid balance and a healthy/normal blood lipid profile by regulating/adjusting the blood lipid levels thus optimizing the blood lipid profile; treating elevated blood lipid levels and high blood cholesterol levels by metabolizing cholesterol and blood lipids; helping to reduce the cholesterol levels in a hyperlipidemic patient; improving dyslipidemia; i.e. the compounds of the formula I may be blood lipids lowering agents.

The compounds of the present invention are particularly intended for the prevention of non-autoimmune type 2 diabetes mellitus in those individuals in high risk to develop this disease, such as individuals with pre-diabetes, impaired glucose tolerance (IGT), or obesity.

Preferred are compounds of the formula I, wherein

R¹=H or OCH₃, preferably R¹=H, or
R³=H, OCH₃ or benzoyloxy, preferably R³=H or OCH₃, or

### R⁵=H, or

R⁷=H, OCH₃ or cinnamoyloxy, R⁸=H, CH₃, OCH₃, preferably R⁸=H or OCH₃, or
R⁷ and R⁸ form together the group O—CH₂—O, or

### R⁹=H, or

### R¹⁰=H.

Even more preferred are compounds of the formula I, wherein R⁴, R⁵ and R⁹ are all hydrogen.

Especially preferred are compounds of the formula I, wherein

R¹=H or OCH₃, preferably R¹=H, and
R³=H, OCH₃ or benzoyloxy, preferably R³=H or OCH₃, and

### R⁵=H, and

R⁷=H, OCH₃ or cinnamoyloxy, R⁸=H, CH₃, OCH₃, preferably R⁸=H or OCH₃,
or R⁷ and R⁸ form together the group O—CH₂—O, and

### R⁹=H, and

### R¹⁰=H.

In other preferred embodiments of the present invention the compound of the formula I is selected from the group consisting of compounds I-1 to I-19, wherein the groups R¹ to R¹⁰ in compounds I-1 to I-19 have the meaning as given in the attached Table 0.

Especially preferred from this group are compounds of the formula I selected from the group consisting of compounds I-2, I-7, I-8, I-9, I-13, I-14, I-15, I-17 and I-19 as defined in Table 0, most preferred are the compounds of the formula I-13, I-14 and I-15 as shown in FIG. 1.

The compound of the formula I-13 is one, in which R¹=R⁴=R⁵=R⁹=H, R³=R⁶=OCH₃, R⁷ and R⁸ form together a group O—CH₂—O and R¹⁰=N-acetyl, N-methyl-2-aminoethyl; the compound of the formula I-14 is one, in which R¹=R⁴=R⁵=R⁶=R⁸, R⁹=R¹⁰=H, R³=OCH₃ and R⁷=cinnamoyloxy; and the compound of the formula I-15 is one, in which R¹=R⁴=R⁵=R⁶=R⁹=R¹⁰=H, R⁷=R⁸=OCH₃ and R³=benzoyloxy.

The term “compound of the formula I” also encompasses any material or extract of a plant containing such a compound of the formula I, preferably in an amount of at least 50 weight-%, more preferably in an amount of at least 70 weight-%, even more preferably in an amount of at least 90 weight-%, based on the total weight of the plant material or extract. The terms “material of a plant” and “plant material” used in the context of the present invention mean any part of a plant.

The compounds of the formula I-13 may be isolated from Papaver pseudo-orientale. The compounds of the formula I-13 and I-15 may be isolated from the poppy plant. The compounds of the formula I-14 and I-15 e.g. may be isolated from Glycyrrhiza glabra (licorice).

The present invention is also directed to the compound of the formula I as defined above for use as inhibitors of glucose uptake such as α-glucosidase inhibitors, as hyperglycemia treating and/or controlling agents, as blood glucose lowering agents, as blood lipids lowering agents, as insulin sensitizing agents, as pancreatic β-cell function improvers, as inhibitors of hepatic glucose production, as insulin mimetics and/or as enhancers of insulin release.

The present invention is further directed to the use of a compound of the formula I as defined above for the manufacture of a composition for the treatment of non-autoimmune type 2 diabetes mellitus and/or syndrome X. The composition is preferably used as inhibitor of glucose uptake such as α-glucosidase inhibitor, as hyperglycemia treating and/or controlling agent, as blood glucose lowering agent, as blood lipid lowering agent, as insulin sensitizing agent, as pancreatic β-cell function improver, as inhibitor of hepatic glucose production, as insulin mimetic and/or as enhancer of insulin release.

A further object of the present invention is a dietary composition containing at least a compound of the formula I as defined and with the preferences given as above.

The term “dietary compositions” comprises any type of (fortified) food, (fortified) (animal) feed and beverages including also clinical nutrition, and also dietary supplements as well as the corresponding additives: food additives, beverage additives, feed additives. Also encompassed is functional food/feed i.e. a food/feed that has been enhanced with vitamins or pharmaceuticals to provide further specific health benefits, as well as a nutraceutical, i.e. a pill or other pharmaceutical product that has nutritional value.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

Another object of the present invention is a pharmaceutical composition containing at least one compound of the formula I as defined and with the preferences given as above and a conventional pharmaceutical carrier.

Beside a pharmaceutically acceptable carrier and at least one compound of the formula I with the definitions of R¹ to R¹⁰ and the preferences as given above, the pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/parenteral/injectable administration.

The dietary and pharmaceutical compositions according to the present invention may be in any galenic form that is suitable for administrating to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be filled into hard or soft shell capsules, whereby the capsules feature e.g. a matrix of (fish, swine, poultry, cow) gelatin, plant proteins or ligninsulfonate. Examples for other application forms are forms for transdermal, parenteral or injectable administration. The dietary and pharmaceutical compositions may be in the form of controlled (delayed) release formulations.

Examples for fortified food are cereal bars, bakery items such as cakes and cookies.

Beverages encompass non-alcoholic and alcoholic drinks as well as liquid preparations to be added to drinking water and liquid food. Non-alcoholic drinks are e.g. soft drinks, sport drinks, fruit juices, lemonades, near-water drinks (i.e. water-based drinks with a low calorie content), teas and milk based drinks. Liquid food are e.g. soups and dairy products.

The compounds of the formula I with the definitions of R¹ to R¹⁰ and the preferences as given above as well as (mixtures of) plant materials and plant extracts containing them, preferably in an amount of at least 50 weight-%, more preferably in an amount of at least 70 weight-%, even more preferably in an amount of at least 90 weight-%, based on the total weight of the plant material or extract, and dietary/pharmaceutical compositions containing them are thus suitable for the treatment of animals including humans.

Therefore, the invention relates to a method for the treatment of non-autoimmune type 2 diabetes mellitus and/or syndrome X in animals including humans, said method comprising the step of administering an effective dose of a compound of the formula I as defined above to animals including humans which are in need thereof.

Animals in the context of the present invention may be mammals including humans.

Preferred examples of mammals beside humans are dogs, cats, guinea pigs, (jack) rabbits, hares, ferrets, horses, and ruminants (cattle, sheep and goat).

For humans a suitable daily dosage of a compound of the formula I with the definitions of R¹ to R¹⁰ and the preferences as given above, for the purposes of the present invention may be within the range from 0.01 mg per kg body weight to 50 mg per kg body weight per day. More preferred may be a daily dosage of 0.1 to 25 mg per kg body weight, and especially preferred may be a daily dosage of 0.3 to 7 mg per kg body weight. The amount of a plant material or plant extract containing such compound of the formula I can be calculated accordingly.

In solid dosage unit preparations for humans, the compound of the formula I with the definitions of R¹ to R¹⁰ and the preferences as given above may be suitably present in an amount from 0.25 mg to 1000 mg, preferably from 2 mg to 200 mg per dosage unit.

In dietary compositions, especially in food and beverages for humans, the compound of the formula I with the definitions of R¹ to R¹⁰ and the preferences as given above may be suitably present in an amount of from 0.5 mg/kg to 100 g/kg, preferably from 5 mg/kg to 10 g/kg, more preferably from 50 mg/kg to 2 g/kg, based upon the total weight of the food or beverage.

In food and drinks in a preferred embodiment of the invention the amount of the compound of the formula I with the definitions of R¹ to R¹⁰ and the preferences as given above may be from 0.7 to 4000 mg per serving.

For animals excluding humans a suitable daily dosage of a compound of the formula I with the definitions of R¹ to R¹⁰ and the preferences as given above for the purposes of the present invention may be within the range from 0.001 mg per kg body weight to 2000 mg per kg body weight per day. More preferred is a daily dosage of 0.01 mg to 1000 mg per kg body weight, and especially preferred is a daily dosage of 0.1 mg to 500 mg per kg body weight.

The present invention is further directed to the compounds of the formula I with the definitions of R¹ to R¹⁰ and the preferences as given above, especially to the compounds I-2, I-7, I-8, I-9, I-13, I-14, I-15, I-17 and I-19 as defined in Table 0, as well as to their use as medicament.

The invention is now further illustrated by the following examples.

### EXAMPLES

The following abbreviations are used:

BW=body weight
DMEM=Dulbecco's Modified eagle Medium
DMSO=dimethylsulfoxide
FBS=Fetal Bovine serum
2-DG=2-deoxyglucose
3[H]-2-DG=tritiated 2-deoxyglucose
HBS=Hanks balanced salt solution

### oil Red O=Solvent Red 27 (CAS-No. 1320-06-5)

PBS=Phosphate buffer solution
OD=optical density
SEM=standard error of the mean
FFA=free fatty acids

### GUA=Glucose Uptake of Adipocytes

### Example 1

### Effect of the Compound of the Formula I-13 on the Glucose Uptake of Adipocytes

C3H10T1/2 cells (ATCC CCL-226) were grown for 5 days to confluence in DMEM supplemented with 10% FBS medium and induced with a mixture of insulin, dexamethasone and 3-isobutyl-1-methylxanthine to differentiate into adipocytes. Nine days after the beginning of induction, cells were treated for 48-h with the compound of the formula I-13 at different concentrations as shown in Table 1. Glucose uptake was determined using radioactive 2-deoxyglucose (10 μM 2-DG in HBS+0.5 μCi/ml of 3[H]-2-DG), measuring glucose uptake in the absence of insulin. Basal glucose uptake was increased by 48-h treatment with the compound of the formula I-13 in a dose-dependent manner (Table 1). As a positive control, ciglitazone was used in the concentration as indicated in Table 1.

### Example 2

### Effect of the Compound of the formula I-15 on Glucose Uptake of Adipocytes

Growing, induction and treatment of C3H10T1/2 cells were exactly as described in Example 1, with the exception that the compound of the formula I-15 at different concentrations was used instead of the compound of the formula I-13. As shown in Table 1, an increase of the basal glucose uptake could be detected.

TABLE 1
Induction of glucose uptake by 48-h treatment with
different compounds (% of control ± SEM)

| | Concentration | |
|---|---|---|
| Compound | [M] | Basal glucose uptake |
| Ciglitazone | 5 × 10⁻⁵ | 496.178 ± 61.86 |
| compound of the formula I-13 | 1 × 10⁻⁶ | 97.6 ± 22.9 |
| | 1 × 10⁻⁵ | 94.58 ± 0.95 |
| | 2.4 × 10⁻⁵ | 128.6 ± 0.89 |
| | 5 × 10⁻⁴ | 194.058 ± 0.05 |
| compound of the formula I-15 | 1 × 10⁻⁶ | 105.37 ± 2.21 |
| | 1 × 10⁻⁵ | 133.28 ± 23.4 |
| | 5 × 10⁻⁵ | 116.17 ± 15.96 |
| | 2 × 10⁻⁴ | 142.84 ± 18.05 |

Control: C3H10T1/2 cells treated for 48 h with DMSO at the same concentration as compound-treated cells and set at 100%

### Example 3

### Effect of the Compound of the formula I-13 on Differentiation of Adipocytes

C3H10T1/2 cells were grown to confluence as described in Example 1, then treated for 10 days with insulin alone (negative control) or with a mixture of insulin and the compound of the formula I-13 at different concentrations (see Table 2), with re-feeding with fresh medium and compounds every 48-h. After 10-days of treatment, the cells were stained with oil Red O as follows: cells were washed 2× in PBS and fixed in 10% formalin at room temperature for 1 h. After removal of formalin, 200 μl of oil Red O staining solution (3:2 mixture of 0.5% w/v oil Red O stock solution and water) was applied to each well. The cells were incubated for 20 min at room temperature, washed twice in 2×PBS and incubated for 10 min with 300 μl of isopropanol/well for oil Red O extraction. Quantification of oil Red O was determined by measuring absorbance at 540 nm (mean OD). Co-treatment of C3H10T1/2 cells with insulin and the compound of the formula I-13 resulted in a higher differentiation of the cells into adipocytes than insulin alone as represented by a higher amount of oil Red O staining (Table 2).

TABLE 2
Induction of adipocyte differentiation by 10-day
treatment with the compound of the formula I-13

| Compound | Mean OD ± SEM |
|---|---|
| Insulin (1 × 10⁻⁷ M) | 0.28 ± 0.03 |
| Insulin (1 × 10⁻⁷ M) + compound | 0.69 ± 0.019 |
| of the formula I-13 (1 × 10⁻⁵ M) | |

### Example 4

### Effect of the Compound of the formula I-14 on Differentiation of Adipocytes

C3H10T1/2 cells were grown and treated as described in Example 4 with the exception that the compound of the formula I-14 was used instead of the compound of the formula I-13. The measurement of adipocyte differentiation using the oil Red O assay was performed as described in Example 4. Co-treatment of C3H10T1/2 cells with insulin and the compound of the formula I-14 resulted in a higher differentiation of the cells into adipocytes than insulin alone (Table 3).

### Example 5

### Effect of Compound of the formula I-15 on Differentiation of Adipocytes

C3H10T1/2 cells were grown and treated as described in Example 4 with the exception that the compound of the formula I-15 was used instead of the compound of the formula I-13. The measurement of adipocyte differentiation using the oil Red O assay was performed as described in Example 4. Co-treatment of C3H10T1/2 cells with insulin and the compound of the formula I-15 resulted in a higher differentiation of the cells into adipocytes than insulin alone (Table 3).

TABLE 3
Induction of adipocyte differentiation by 10-day
treatment with the compound of the formula I-14
or the compound of the formula I-15.

| Compound | Mean OD ± SEM |
|---|---|
| Insulin (1 × 10⁻⁷ M) | 0.28 ± 0.030 |
| Insulin (1 × 10⁻⁷ M) + compound | 0.45 ± 0.037 |
| of the formula I-14 (1 × 10⁻⁵ M) | |
| Insulin (1 × 10⁻⁷ M) + compound | 0.53 ± 0.025 |
| of the formula I-15 (1 × 10⁻⁵ M) | |
| Insulin (1 × 10⁻⁷ M) + compound | 0.53 ± 0.017 |
| of the formula I-15 (5 × 10⁻⁵ M) | |
| Insulin (1 × 10⁻⁷ M) + compound | 0.34 ± 0.087 |
| of the formula I-15 (2 × 10⁻⁴ M) | |

### Example 6

### Effect of the Compound of the Formula I-13 on Glucose Tolerance

The efficacy of the compound of the formula I-13 on glucose tolerance was tested in a 14-day study in C57BLKS/J db/db mice (n=10/group), a model of late type 2 diabetes mellitus with severe hyperglycemia.

Male db/db mice were obtained from Jackson Laboratory (Bar Harbor, Me., USA). Adult mice aged 8 weeks were used in the experiment. Mice were housed individually in plastic cages with bedding and allowed free access to standard rodent food and tap water. The animal rooms were controlled for temperature (24° C.), humidity (55%), and light (12-h light-dark cycle). The animals were randomized in two groups and the compound of the formula I-13 was administered orally to one of the groups for 14 days at a dose of 200 mg/kg BW/day. After 14 days of treatment the concentration of glucose was determined in blood from fed animals, i.e., animals which were not restricted from food. After a period of 10 days of treatment an oral glucose tolerance test (OGTT) was performed. For the OGTT mice were fasted overnight and then a 1-g glucose/kg BW solution was orally administered. Blood samples were taken before and 15, 30, 45, 60, 90, 120, 150, 180 min after the glucose challenge for determination of blood glucose levels and then the area under the curve (AUC) was determined. Blood glucose was measured by a glucose analyzer (Glucotrend Premium, Roche Diagnostics, Rotkreuz, Switzerland). The blood glucose levels and AUC for the OGTT measurement are given in Table 4. The glucose and the free fatty acid (FFA) levels of fed animals (see above) were lowered after 14 days of treatment with the compound of the formula I-13. After 10 days of treatment with the compound of the formula I-13 the glucose levels of fasted animals, i.e., animals with an overnight fasting (see above) were decreased as compared to the untreated control group. During the OGTT test the blood glucose levels in the animals treated with the compound of the formula I-13 were lower at all time points when compared with the control group. Thus, the compound of the formula I-13 significantly reduced the glucose AUC of an OGTT (1 g glucose/kg body weight) on day 10.

TABLE 4
Blood glucose level in db/db mice treated
with the compound of the formula I-13

| | Blood Glucose | | | |
|---|---|---|---|---|
| | Fasted | Fed | Glucose | FFA |
| | (mg/dl) | (mg/dl) | AUC | (mg/dl) |
| Control | 175 | 859 | 69673 | 15.95 |
| compound of the formula I-13 | 135 | 771 | 54295 | 12.38 |
| (200 mg/kg BW/day) | | | | |

TABLE 0
Compounds of the formulae I-1 to I-23.

| No. | R-1 | R-3 | R-4 | R-5 | R-6 | R-7 | R-8 | R-9 | R-10 |
|---|---|---|---|---|---|---|---|---|---|
| I-1 | H | CH₃ | H | H | OCH₃ | H | H | OCH₃ | H |
| I-2 | H | CH₃ | H | H | OCH₃ | H | OCH₃ | H | H |
| I-3 | H | OH | H | H | H | H | OCH₃ | H | H |
| I-4 | H | H | H | H | H | H | OH | H | H |
| I-5 | H | H | H | H | H | CH₃ | H | H | H |
| I-6 | CH₃ | H | H | H | H | H | H | H | H |
| I-7 | H | H | H | H | H | H | CH₃ | H | H |
| I-8 | H | CH₃ | H | H | H | H | OCH₃ | H | H |
| I-9 | H | H | H | H | H | H | CH₃ | H | H |
| I-10 | OCH₃ | OH | H | H | H | H | OH | H | H |
| I-11 | OCH₃ | OCH₃ | H | OH | H | H | H | H | H |
| I-12 | OCH₃ | OCH₃ | H | OH | H | H | H | H | H |
| I-13 | H | OCH₃ | H | H | OCH₃ | O—CH₂—O | | H | N-acetyl, |
| | | | | | | | | | N-methyl-2- |
| | | | | | | | | | aminoethyl |
| I-14 | H | OCH₃ | H | H | H | cinnamoyloxy | H | H | H |
| I-15 | H | benzoyloxy | H | H | H | OCH₃ | OCH₃ | H | H |
| I-16 | H | OCH₃ | H | H | OCH₃ | H | H | OCH₃ | H |
| I-17 | H | H | H | H | OCH₃ | O—CH₂—O | | H | N-acetyl, |
| | | | | | | | | | N-methyl-2- |
| | | | | | | | | | aminoethyl |
| I-18 | H | OCH₃ | H | H | H | H | OH | H | H |
| I-19 | H | OCH₃ | H | H | H | (3,4,5-trimeth- | H | H | H |
| | | | | | | oxy)benzoyloxy | | | |

1. Compounds of the formula I, 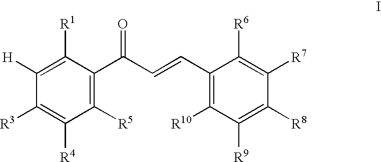
wherein R¹ is H, CH₃ or OCH₃;
R³=H, OH, CH₃, OCH₃ or benzoyloxy;
R⁴=H;
R⁵H or OH;
R⁶H or OCH₃;
R⁷H, CH₃, OCH₃, cinnamoyloxy or (3,4,5-trimethoxy)-benzoyloxy;
R⁸=H, OH, CH₃ or OCH₃;
or R⁷ and R⁸ form together a group O—CH₂—O;
R⁹H or OCH₃;
R¹⁰=H or N-acetyl, N-methyl-2-aminoethyl;
for use as medicament for the treatment of non-autoimmune type 2 diabetes mellitus and/or syndrome X.

2. Compound of the formula I according to claim 1, wherein the compound is selected from the group of compounds I-1 to I-19, wherein the groups R¹ to R¹⁰ in compounds I-1 to I-19 have the following meaning:

| No. | R-1 | R-3 | R-4 | R-5 | R-6 | R-7 | R-8 | R-9 | R-10 |
|---|---|---|---|---|---|---|---|---|---|
| I-1 | H | CH3 | H | H | OCH3 | H | H | OCH3 | H |
| I-2 | H | CH3 | H | H | OCH3 | H | OCH3 | H | H |
| I-3 | H | OH | H | H | H | H | OCH3 | H | H |
| I-4 | H | H | H | H | H | H | OH | H | H |
| I-5 | H | H | H | H | H | CH3 | H | H | H |
| I-6 | CH3 | H | H | H | H | H | H | H | H |
| I-7 | H | H | H | H | H | H | CH3 | H | H |
| I-8 | H | CH3 | H | H | H | H | OCH3 | H | H |
| I-9 | H | H | H | H | H | H | CH3 | H | H |
| I-10 | OCH3 | OH | H | H | H | H | OH | H | H |
| I-11 | OCH3 | OCH3 | H | OH | H | H | H | H | H |
| I-12 | OCH3 | OCH3 | H | OH | H | H | H | H | H |
| I-13 | H | OCH3 | H | H | OCH3 | O—CH2-O | | H | N-acetyl, N |
| | | | | | | | | | methyl-2- |
| | | | | | | | | | aminoethyl |
| I-14 | H | OCH3 | H | H | H | cinnamoyl- | H | H | H |
| | | | | | | oxy | | | |
| I-15 | H | benzoyloxy | H | H | H | OCH3 | OCH3 | H | H |
| I-16 | H | OCH3 | H | H | OCH3 | H | H | OCH3 | H |
| I-17 | H | H | H | H | OCH3 | O—CH2-O | | H | N-acetyl, N |
| | | | | | | | | | methyl-2- |
| | | | | | | | | | aminoethyl |
| I-18 | H | OCH3 | H | H | H | H | OH | H | H |
| I-19 | H | OCH3 | H | H | H | (3,4,5-trimeth- | H | H | H |
| | | | | | | oxy)-benzoyloxy | | | |

3. Compound of the formula I according to claim 2, wherein the compound is selected from the group consisting of compounds I-2, I-7, I-8, I-9, I-13, I-14, I-15, I-17 and I-19 as defined in claim 2.

4. Compound of the formula I according to claim 1, wherein it is selected from the group of compounds I-13 to I-15, wherein the compound of the formula I-13 is one, in which
R¹=R⁴=R⁵=R⁹=H, R³=R⁶=OCH₃, R⁷ and R⁸ form together a group O—CH₂—O and R¹⁰=N-acetyl, N-methyl-2-aminoethyl;
the compound of the formula I-14 is one, in which R¹=R⁴=R⁵=R⁶=R⁸R⁹=R¹⁰=H, R³=OCH₃ and R⁷=cinnamoyloxy; and
the compound of the formula I-15 is one, in which R¹=R⁴=R⁵=R⁶=R⁹=R¹⁰=H, R⁷=R⁸=OCH₃ and R³=benzoyloxy.

5. Compound of the formula I according to claim 1, wherein
R¹=H or OCH₃, or
R³=H, OCH₃ or benzoyloxy, or
R⁵=H, or
R⁷=H, OCH₃ or cinnamoyloxy, R⁸=H, CH₃, OCH₃, or R⁷ and R⁸ form together the group O—CH₂—O, or
R⁹=H

6. Compound of the formula I according to claim 1, wherein R⁴, R⁵ and R⁹ are all hydrogen.

7. Compound of the formula I according to claim 1, wherein
R¹=H or OCH₃, and
R³=H, OCH₃ or benzoyloxy, and
R⁵=H, and
R⁷=H, OCH₃ or cinnamoyloxy, R⁸=H, CH₃, OCH₃, or R⁷ and R⁸ form together the group O—CH₂—O, and
R⁹=H.

8. Compound of the formula I according to claim 1, wherein
R¹=H or OCH₃, preferably =H, or
R³=H or OCH₃, or
R⁸=H or OCH₃, or
R¹⁰=H.

9. Compound of the formula I according to claim 1, wherein
R¹=H or OCH₃, preferably =H, and
R³=H or OCH₃, and
R⁸=H or OCH₃, and
R¹⁰=H.

10. The compound of the formula I as defined in claim 1, as inhibitor of glucose uptake such as α-glucosidase inhibitor, as hyperglycemia treating and/or controlling agent, as blood glucose lowering agent, as blood lipid lowering agent, as insulin sensitizing agent, as pancreatic β-cell function improver, as inhibitor of hepatic glucose production, as insulin mimetic and/or as enhancer of insulin release.

11. A compound of the formula I as defined in claim 1, for the manufacture of a composition for the treatment of non-autoimmune type 2 diabetes mellitus and/or syndrome X.

12. The composition according to claim 11, wherein the composition is used as inhibitor of glucose uptake such as α-glucosidase inhibitor, as hyperglycemia treating and/or controlling agent, as blood glucose lowering agent, as blood lipid lowering agent, as insulin sensitizing agent, as pancreatic β-cell function improver, as inhibitor of hepatic glucose production, as insulin mimetic and/or as enhancer of insulin release.

13. A dietary composition containing at least a compound of the formula I as defined in claim 1.

14. The dietary composition according to claim 13, wherein the compound of the formula I is selected from the group consisting of compounds of the formula I-13 to I-15.

15. A pharmaceutical composition containing at least one compound of the formula I as defined in claim 1, and a conventional pharmaceutical carrier.

16. The pharmaceutical composition according to claim 15, wherein the compound of the formula I is selected from the group consisting of compounds of the formula I-13 to I-15.

17. A method for the treatment of non-autoimmune type 2 diabetes mellitus and/or syndrome X in animals including humans, said method comprising the step of administering an effective dose of a compound of the formula I as defined in claim 1, to animals including humans which are in need thereof.

18. The method according to claim 17, wherein the animal is a human, a pet animal or a farm animal.

19. Compounds of the formula I as defined in claim 1.

20. Compounds of the formula I as defined in claim 1, for use as medicament.

21. Compounds I-2, I-7, I-8, I-9, I-13, I-14, I-15, I-17 and I-19 as defined in claim 2.

22. Compounds I-2, I-7, I-8, I-9, I-13, I-14, I-15, I-17 and I-19 as defined in claim 2 for use as medicament.
